(19) United States
(12) Patent Application Publication
(10) Pub. No.: US 2025/0334568 A1
(43) Pub. Date: Oct. 30, 2025

(54) IN VIVO-COMPARABLE IN VITRO ASSAY FOR LUNG EPITHELIAL BARRIER INJURY

(71) Applicants: U.S. ENVIRONMENTAL PROTECTION AGENCY, Washington, DC (US); THE UNIVERSITY OF NORTH CAROLINA AT CHAPEL HILL, Chapel Hill, NC (US)

(72) Inventors: Shaun David MCCULLOUGH, Pittsboro, NC (US); Alysha SIMMONS, Cary, NC (US)

(21) Appl. No.: 19/255,811

(22) Filed: Jun. 30, 2025

Related U.S. Application Data
(62) Division of application No. 18/083,201, filed on Dec. 16, 2022.
(60) Provisional application No. 63/352,477, filed on Jun. 15, 2022.

(51) Int. Cl. G01N 33/50; C12N 5/071; G01N 21/64; G01N 33/58

(52) U.S. Cl. CPC G01N 33/5044; C12N 5/0688; G01N 21/6428; G01N 33/582; G01N 2021/6439

(57) ABSTRACT

A method of testing a test substance in an in vitro model of a human tracheobronchial respiratory tract, includes: providing the in vitro model including a cell culture including airway epithelial cells (AECs), a basolateral compartment below the AECs and an apical compartment above the AECs, wherein the AECs form a barrier between the basolateral AND apical compartments; adding the test substance to the apical and/or basolateral compartment; adding a tracer to the basolateral compartment, which is fluorescent, has a molecular weight within 5 kDa of human albumin and is added before, during or after adding the test substance; incubating the cell culture system in a presence of the tracer; collecting at least one sample from the apical compartment; and measuring a fluorescence thereof to determine an effect of the test substance on the AECs. A kit is also disclosed.

### CROSS-REFERENCE TO RELATED APPLICATIONS

This application is a divisional application of U.S. patent application Ser. No. 18/083,201, filed Dec. 16, 2022, which claims the benefit under 35 U.S.C. § 119 (e) of U.S. Provisional Patent App. No. 63/352,477, filed Jun. 15, 2022, the contents of which applications are incorporated herein by reference in their entireties for all purposes.

### GOVERNMENT RIGHTS

The United States government has certain rights in the invention because an employee of the United States Environmental Protection Agency is a co-inventor, and the invention was supported by general EPA research funds.

### BACKGROUND OF THE INVENTION

### 1. Field of Invention

This invention relates to in vitro models of biological systems, and particularly to an in vitro assay useful for conducting a respiratory hazard assessment of a test substance.

### 2. Description of Related Art

The Frank R. Lautenberg Chemical Safety for the 21st Century Act, an amendment to the Toxic Substances Control Act (TSCA), mandates that the United States Environmental Protection Agency (EPA) use the best available science while also promoting the use of non-animal alternative testing methodologies for the evaluation of chemical safety. Meeting this mandate requires the use of “new approach methodologies” (NAMs) including advanced in vitro cell-based models and assays that reflect in vivo human biology and adverse health outcomes.

Inhalation is one of the three routes of toxicant exposure and the efforts of the EPA, as well as those of the broader stakeholder community, to incorporate NAMs into inhaled chemical safety assessment have focused on the use of in vitro models of the human tracheobronchial respiratory tract that are constructed with cells derived directly from human donors (referred to as “primary cells”). These models strive to recapitulate key features of the human tracheobronchial respiratory tract in vivo including its function as a barrier between inhaled chemicals/materials and the underlying lung tissue. Disruption of the integrity of this bronchial epithelial barrier is indicated by the leakage of the protein albumin into the respiratory tract, which occurs following the inhalation of toxic chemicals.

Primary human bronchial epithelial cell cultures, and primary human cell cultures from other regions of the respiratory tract (airway epithelia cells), can be grown on porous membranes and differentiated in vitro in air-liquid interface culture conditions to generate a physiologically-relevant cell culture system 10 for the evaluation of inhaled chemical toxicity and pharmaceutical efficacy (FIG. 1). Given their close representation to the in vivo human airway epithelial barrier, these in vitro models are currently the preferred system for chemical safety evaluation and early pharmaceutical development of inhaled agents. The impact of inhaled materials on airway epithelial barrier integrity in in vitro systems is a key endpoint in chemical safety assessment and currently most commonly evaluated using trans-epithelial electrical resistance (TEER). TEER is a method that infers changes in barrier integrity based on differences in the electrical resistance of the airway epithelial cell layer. The second, and less common, method involves measurement of the apical to basolateral (“downward diffusion”) translocation of low molecular weight fluorescent molecules (often 0.4-20 kilodaltons (kDa)) in a fluorescent tracer solution 20 through an airway epithelial cell barrier 12 formed by an in vitro cell-based cell culture system 10 (FIG. 2C).

TEER measurements reflect the relative electrical resistance of epithelial cell barriers and can be a sensitive method for detecting changes in epithelial barrier integrity that are reflective of airway injury. However, TEER does not provide information regarding fluid flux or relative size of pores opened in the epithelial barrier. TEER is also not directly comparable to airway injury assays used in in vivo human studies.

The downward diffusion approach requires that the airway epithelial cell layer be re-submerged in the fluorescent tracer solution. Doing so abolishes the air-liquid interface culture condition, which is a key feature of differentiated primary human airway epithelial cell cultures and the application of liquid on air-liquid interface cultures alone reduces airway epithelial barrier integrity. Additionally, while albumin leakage into the respiratory tract following airway injury occurs from the basolateral to apical direction in vivo, the downward diffusion approach evaluates the movement of tracer molecules in the apical to basolateral direction. Further, the downward diffusion method is generally conducted using fluorescent molecules with a significantly lower molecular weight than the albumin protein that is measured in vivo.

Examples of such diffusion methods include the Evans blue-bovine albumin diffusion method, which evaluates the diffusion of bovine serum albumin that has been non-covalently bound to Evans blue dye. This method was used in a downward diffusion application to evaluate the effect of histamine exposure on an in vitro model of the blood-brain barrier. The disadvantages of this method are: (1) it relies on a colorimetric dye, which is less sensitive than fluorescent dyes; (2) the binding of Evans blue dye to albumin is reversible and free dye (0.9 kDa) would be able to traverse small pores in an epithelial barrier, and/or through cells with damaged cell membranes, and confound the interpretation of barrier integrity assay results; and (3) it relies on albumin that has been isolated from bovine serum, which is not 100% pure and may contain contaminants that could impact epithelial barrier integrity and assay performance. See Deli et al. (1995). “Histamine induces a selective albumin permeation through the blood-brain barrier in vitro.” Inflamm Res. 44 Suppl 1: S56-7. DOI: 10.1007/BF01674394.

Plateel et al. (1997) Hypoxia dramatically increases the nonspecific transport of blood-borne proteins to the brain. J Neurochem. 68 (2): 874-7. DOI: 10.1046/j. 1471-4159.1997.68020874.x discloses a radioisotope-labeled albumin diffusion method. The method evaluates the diffusion of albumin that has been labeled with a radioactive isotope across a cellular barrier. The referenced example utilized tritiated (3H) bovine albumin. This method has been most frequently used to evaluate the permeability of in vitro models of the blood-brain barrier. The primary disadvantage of this method is that it involves the use of radioactive isotopes, which require extensive safety training and protocols, tracking of usage and waste generation, and specialized synthesis of labeled materials. Additionally, given efforts to reduce the utilization of radioisotopes in research, the equipment required to quantify samples may not be readily available.

Annunziata et al. (1998). “HIV-1 gp120 increases the permeability of rat brain endothelium cultures by a mechanism involving substance P.” AIDS. 12 (18): 2377-85. DOI: 10.1097/00002030-199818000-00006 discloses a biotin-conjugated albumin diffusion method, which evaluates the diffusion of bovine serum albumin that was covalently conjugated to biotin across rat brain microvessel cultures. The diffusion of biotin-conjugated albumin was quantified by treatment of samples with streptavidin-conjugated peroxidase (streptavidin binds non-covalently to biotin) and subsequent detection of peroxidase enzyme activity. The disadvantages of this method are: (1) it relies on the quantification of the conversion of a substrate (o-phenylenediamine) to product by peroxidase by spectrophotometry, which is less sensitive than fluorescence quantification and the activity of the peroxidase enzyme may be influenced by test agents being evaluated; and (2) it relies on the purification of both albumin and peroxidase, which is not 100% pure and may contain contaminants that could impact epithelial barrier integrity and assay performance. In addition, biotin is a naturally-occurring post-translational modification, which may confound the assay.

Chen et al. (2014). “Pulmonary permeability assessed by fluorescent-labeled dextran instilled intranasally into mice with LPS-induced acute lung injury.” PLOS ONE. 9 (7): e101925. DOI: 10.1371/journal.pone.0101925 discloses the intranasal installation of FITC-conjugated low molecular weight (3 and 4 kDa) dextran in mice after treatment-induced lung injury. Treatment-induced injury severity was assessed by the relative amount of fluorescent signal detected in peripheral blood of treated and control mice. The disadvantages of this method are: (1) it is not an in vitro method; (2) it relies on the in vivo equivalent of downward diffusion to quantify changes in epithelial barrier integrity; and (3) it relies on the diffusion of fluorescent dextrans that are of a significantly lower molecular weight (3 and 4 kDa) than albumin (67 kDa).

All references cited herein are incorporated herein by reference in their entireties.

### BRIEF SUMMARY OF THE INVENTION

Accordingly, a first aspect of the invention is a method of testing a test substance in an in vitro model of a human tracheobronchial respiratory tract, said method comprising:
providing the in vitro model comprising a cell culture comprising airway epithelial cells, a basolateral compartment below the airway epithelial cells and an apical compartment above the airway epithelial cells, wherein the airway epithelial cells form a barrier between the basolateral compartment and the apical compartment;
adding the test substance to the apical and/or basolateral compartment;
adding a tracer to the basolateral compartment, wherein the tracer is fluorescent, has a molecular weight within 5 kDa of a molecular weight of human albumin and is added to the basolateral compartment before, during or after adding the test substance to the apical and/or basolateral compartment;
incubating the cell culture system in a presence of the tracer;
collecting at least one sample from the apical compartment of the cell culture; and
measuring a fluorescence of the at least one sample to determine an effect of the test substance on the airway epithelial cells.

In certain embodiments, the tracer is a fluorescent molecule.

In certain embodiments, the tracer is a fluorescein isothiocyanate-conjugated dextran having a molecular weight of 70 kDa, or a fluorescein isothiocyanate-conjugated albumin.

In certain embodiments, multiple samples are collected over a period of time.

In certain embodiments, the method is conducted at 37° C. under 5% CO₂ with relative humidity >80.

In certain embodiments, the effect tested is an integrity of the barrier of airway epithelial cells.

In certain embodiments, a hazardousness of the test substance is directly correlated with the fluorescence measured.

In certain embodiments, the effect tested is an exogenous expression of a genetic modification of the airway epithelial cells by the test substance.

In certain embodiments, an air-liquid interface culture condition is maintained throughout the method.

In certain embodiments, a liquid-liquid interface culture condition is maintained for at least a portion of the method.

In certain embodiments, the test substance is an inhalable material selected from the group consisting of gases, vapors, aerosols, particulates and biological materials.

A second aspect of the invention is a kit for practicing the method of the invention, said kit comprising:
a first container containing the tracer which is fluorescent and has a molecular weight within 5 kDa of the molecular weight of human albumin; and
a second container containing a positive control.

In certain embodiments, the positive control is at least one member selected from the group consisting of Cinnamaldehyde, Maltol, Acetoin, Ozone, Lipopolysaccharide (LPS), Tumor necrosis factor alpha (TNF-α) and Interferon gamma (IFN-γ).

In certain embodiments, the kit further comprises a third container containing a negative control.

In certain embodiments of the kit, the tracer is a fluorescein isothiocyanate-conjugated dextran having a molecular weight of 70 kDa, or a fluorescein isothiocyanate-conjugated albumin.

In certain embodiments, the kit further comprises airway epithelial cells.

### BRIEF DESCRIPTION OF THE DRAWINGS

The invention will be described in conjunction with the following drawings in which like reference numerals designate like elements and wherein:

FIG. 1 is a schematic representation of a method of evaluating inhaled chemical toxicity and pharmaceutical efficacy using an in vitro cell culture system and method of the prior art.

FIG. 2 is a schematic representation of a method of evaluating inhaled chemical toxicity and pharmaceutical efficacy using an embodiment of the in vitro cell culture system and method of the instant invention.

### DETAILED DESCRIPTION OF PREFERRED EMBODIMENTS OF THE INVENTION

The invention comprises a simple in vitro assay that is directly representative of leakage of the protein albumin into the respiratory tract, which is used to assess respiratory tract damage in in vivo human and animal exposure studies. The invention has basic and applied science applications in inhalation toxicology, respiratory biology, pathology, and pharmacology across academic, government, and industry sectors. The invention supports the efforts of the EPA to implement human-relevant NAMs for the safety evaluation of inhaled chemicals.

The invention was prompted by an interest in developing assays for use in in vitro primary human bronchial epithelial cell air-liquid interface culture models that would be directly comparable to assays used to evaluate the effects of inhaled chemicals on airway epithelial barrier integrity in in vivo human and animal exposure studies.

The invention evaluates the movement of a tracer in fluorescent tracer solution 20 that is similar in molecular weight to human albumin (67 kDa) in the basolateral to apical direction (arrow 22 in FIG. 2(C)) across a barrier of bronchial epithelial cells 12.

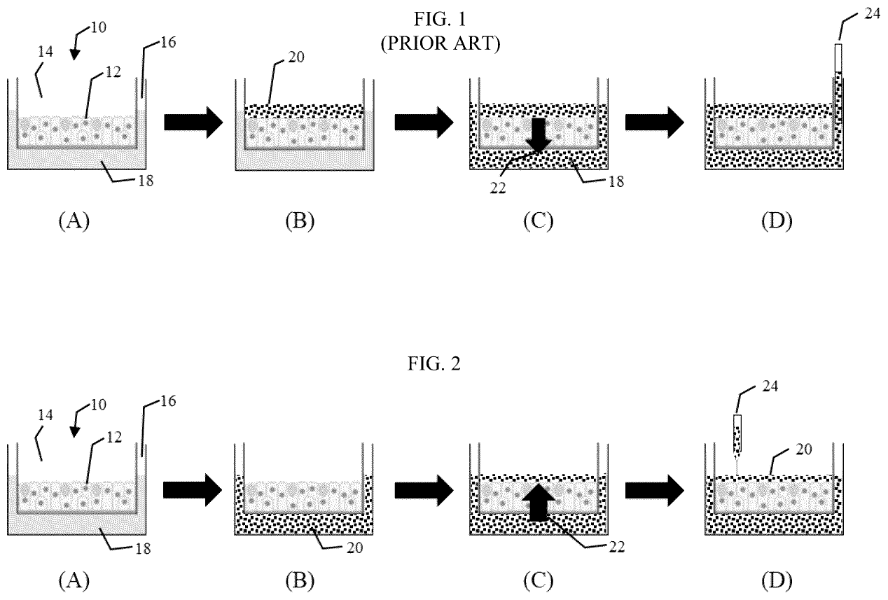

Referring to FIGS. 1(A) and 2(A), cell culture system 10 is provided, which comprises airway epithelial cells 12 on the bottom of apical compartment 14, and basolateral compartment 16 below and lateral to apical compartment 14. Culture medium 18 is in basolateral compartment 16 and is in fluid communication with airway epithelial cells 12 in apical compartment 14.

Airway epithelial cells suitable for use in the invention can be, e.g., primary cells or a cell line. Certain cell lines can be used at air-liquid interface and thus would be applicable in that context.

A test substance is added to the apical and/or basolateral compartment. The test substance is preferably inhalable and selected from the group consisting of gases, vapors, aerosols, particulates and biological materials. The test substance can be applied to the apical and/or basolateral compartment by any desired exposure method including but not limited to direct application of liquid, and air-liquid interface exposure. In certain embodiments, a cell culture exposure system may be used, such as the system disclosed by Zavala et al. “A new cell culture exposure system for studying the toxicity of volatile chemicals at the air-liquid interface.” Inhalation toxicology vol. 30,4-5 (2018): 169-177. doi: 10.1080/08958378.2018.1483983. Alternatively, commercial exposure systems available from, e.g., VitroCell (see www.vitrocell.com/inhalation-toxicology/exposure-systems) and CelTox (see www.medtecbiolab.com/products/in-vitro-systems) may be used.

Fluorescent tracer solution 20 is added to basolateral compartment 20 in the embodiment of the invention shown in FIG. 2(B). On the other hand, in the conventional method shown in FIG. 1(B), fluorescent tracer solution 20 is added to apical compartment 14 on top of airway epithelial cells 12.

In the incubation step depicted in FIG. 2(C), fluorescent tracer solution 20 is given time to translocate from basolateral compartment 16 to apical compartment 14 by active transportation or passive paracellular translocation. This upward translocation is represented by arrow 22. The amount of time can be, e.g., 1-72 hours or 2-48 hours or 4-24 hours in certain non-limiting embodiments.

In the conventional incubation step of FIG. 1(C), fluorescent tracer solution 20 is given time to translocate from apical compartment 14 to basolateral compartment 16, as represented by arrow 22.

FIG. 2(D) shows a collecting step, wherein fluorescent tracer solution 20, which has penetrated the barrier of airway epithelial cells 12, is collected by sampling means 24 from apical compartment 14 and then analyzed for fluorescence. A single sample can be collected, multiple substantially simultaneous samples can be collected or multiple samples can be collected over an extended period of time (e.g., 1-72 hours or 2-48 hours or 4-24 hours in certain non-limiting embodiments).

In the conventional incubation step of FIG. 1(C), fluorescent tracer solution 20 is collected from basolateral compartment 16.

The tracer is preferably a fluorescent molecule, and is more preferably a fluorescent dextran or albumin molecule. In certain embodiments, the tracer is a fluorescein isothiocyanate-conjugated 70 kDa dextran (“70 kDa-FITC dextran”) or a fluorescein isothiocyanate-conjugated albumin (“FITC-albumin”). The tracer is added to cell culture medium 18 in basolateral compartment 16, thus maintaining the air-liquid interface culture condition in a preferred embodiment of the invention. The relative integrity of an airway epithelial cell barrier is assessed by measuring fluorescent signal intensity present in an apical wash with saline, or other biologically relevant buffer solution, following incubation. When added to the basolateral medium beneath a differentiated primary human airway epithelial barrier in air-liquid interface conditions with an intact, functional epithelial barrier, preferred tracer molecules will exhibit minimal, or no, translocation from the basolateral compartment to the apical compartment by active transportation or passive paracellular translocation. Alternatively, damage to the cell barrier results in the formation of paracellular gaps that would allow for leakage, or increased leakage, of the tracer molecules from the basolateral compartment to the apical surface of the culture (upward translocation). The relative amount of tracer upward translocation is proportional to the severity of damage (greater damage results in more/larger paracellular gaps and thus increased tracer translocation). Translocated tracer molecules are collected by washing of the apical surface of cultures being evaluated with a suitable buffer solution and quantified using fluorescence intensity quantification. This approach also allows minimal disruption to air-liquid interface culture conditions, allowing for the longitudinal evaluation of barrier integrity with washes of the apical surface of cultures over time, and recapitulating similar bronchoalveolar lavage fluid (BALF) collection protocols used in in vivo human and animal exposure studies.

Use of the invention on live cells can be conducted over a short period of time (e.g., 1, 5, 10, 20, 30 minutes) at room temperature (e.g., 20-22° C.) or tissue culture conditions (i.e., humidified environment at 37° C. with 5% carbon dioxide (CO₂)). Longer-term use (e.g., over 30 minutes to many hours) should be conducted under tissue culture conditions needed to maintain a healthy, living cell population. It is also possible to conduct the method under refrigerated conditions (e.g., 2-6° C. or 3-5° C.). Preferred assay conditions are tissue culture conditions. Thus, e.g., it is preferred to conduct the assay at a temperature of 37° C. under 5% CO₂ with relative humidity >80%.

The inventive method overcomes several limitations and disadvantages of existing assays/methods for evaluating barrier integrity in in vitro primary human bronchial epithelial cell air-liquid interface systems for both research and high-throughput chemical safety and efficacy testing. Advantages of preferred embodiments of the invention include at least one of the following.

The method evaluates the movement of a tracer that is similar in molecular weight to human albumin, which is an analyte evaluated as an indicator of airway injury and reduced epithelial barrier integrity in in vivo exposure studies, in the basolateral to apical direction across an airway epithelial cell barrier (see FIG. 2D).

The method is conducted in a cell culture comprising the airway epithelial cell barrier between a basolateral compartment and an apical compartment. The cell culture can be provided by methods known to those skilled in the art. See, e.g., Fulcher et al. “Well-differentiated human airway epithelial cell cultures.” in Human cell culture protocols, pp. 183-206. Humana Press, 2005.

The tracer is added to the basolateral cell culture medium, thus maintaining the air-liquid interface culture condition.

The assay allows measurement of changes in epithelial barrier permeability over longer periods of time while maintaining air-liquid interface culture conditions.

In certain embodiments, the tracer does not require purification of proteins from other organisms, which will introduce potential confounding contaminants.

The use of a fluorescent tracer, such as 70 kDa-FITC dextran, provides greater assay sensitivity than colorimetric methods and avoids the use of a radioactive analyte.

The invention can also be used to study the exogenous expression of a genetic modification of the airway epithelial cells by a test substance. Non-limiting examples of this include the exogenous expression of recombinant DNA, gene silencing (e.g., RNAi), or alteration of endogenous genes (e.g., CRISPR/Cas9-based editing).

While the invention has been described in detail and with reference to specific examples thereof, it will be apparent to one skilled in the art that various changes and modifications can be made therein without departing from the spirit and scope thereof.

What is claimed is:

1. A method of testing a test substance in an in vitro model of a human tracheobronchial respiratory tract, said method comprising:
providing the in vitro model comprising a cell culture comprising airway epithelial cells, a basolateral compartment below the airway epithelial cells and an apical compartment above the airway epithelial cells, wherein the airway epithelial cells form a barrier between the basolateral compartment and the apical compartment;
adding the test substance to the apical and/or basolateral compartment;
adding a tracer to the basolateral compartment, wherein the tracer is fluorescent, has a molecular weight within 5 kDa of a molecular weight of human albumin and is added to the basolateral compartment before, during or after adding the test substance to the apical and/or basolateral compartment;
incubating the cell culture system in a presence of the tracer;
collecting at least one sample from the apical compartment of the cell culture; and
measuring a fluorescence of the at least one sample to determine an effect of the test substance on the airway epithelial cells.

2. The method of claim 1, wherein the tracer is a fluorescent molecule.

3. The method of claim 1, wherein the tracer is a fluorescein isothiocyanate-conjugated dextran having a molecular weight of 70 kDa, or a fluorescein isothiocyanate-conjugated albumin.

4. The method of claim 1, wherein multiple samples are collected over a period of time.

5. The method of claim 1, wherein the method is conducted at 37° C. under 5% CO₂ with relative humidity >80.

6. The method of claim 1, wherein the effect tested is an integrity of the barrier of airway epithelial cells.

7. The method of claim 6, wherein a hazardousness of the test substance is directly correlated with the fluorescence measured.

8. The method of claim 1, wherein the effect tested is an exogenous expression of a genetic modification of the airway epithelial cells by the test substance.

9. The method of claim 1, wherein an air-liquid interface culture condition is maintained throughout the method.

10. The method of claim 1, wherein a liquid-liquid interface culture condition is maintained for at least a portion of the method.

11. The method of claim 1, wherein the test substance is an inhalable material selected from the group consisting of gases, vapors, aerosols, particulates and biological materials.
